SEQUENCE LISTING (1 sequence)

SEQ ID NO: 1 - protein, 5 aa, Homo sapiens
LYDVA

(19) United States
(12) Patent Application Publication
(10) Pub. No.: US 2024/0165208 A1
(43) Pub. Date: May 23, 2024

(54) Mutant KRAS-Targeting Peptide and Use Thereof

(71) Applicant: GJMEDI INC., Busan (KR)

(72) Inventors: Se Bok Jang, Busan (KR); Mi Suk Jeong, Busan (KR); Chang Woo Han, Busan (KR)

(21) Appl. No.: 18/030,222

(22) PCT Filed: Sep. 29, 2021
(86) PCT No.: PCT/KR2021/013302

§ 371 Date: Apr. 4, 2023

(30) Foreign Application Priority Data
Oct. 8, 2020 (KR) 10-2020-0130300

(51) Int. Cl. A61K 38/46; A61K 9/00; A61K 9/16; A61K 9/20; A61K 9/48; A61K 47/26; A61P 35/00

(52) U.S. Cl. CPC A61K 38/465; A61K 9/0014; A61K 9/1611; A61K 9/1623; A61K 9/2013; A61K 9/2018; A61K 9/2059; A61K 9/4858; A61K 9/4866; A61K 47/26; A61P 35/00

(57) ABSTRACT

The present invention relates to a mutant KRAS-targeting peptide and, more specifically, to: a composition for prevention, amelioration or treatment of cancer, comprising an anticancer peptide; and an anticancer adjuvant composition. The mutant KRAS-targeting peptide according to the present invention not only forms a conjugate with mutant KRAS, but also has an effect of inhibiting cancer by blocking the activity of RAS, and thus can be used in various ways in the fields of cancer prevention and treatment.

### TECHNICAL FIELD

The present invention relates to a mutant KRAS-targeting peptide, and more particularly, to a composition for prevention, amelioration, or treatment of cancer including the anticancer peptide, and an anticancer adjuvant composition.

### REFERENCE TO AN ELECTRONIC SEQUENCE LISTING

The contents of the electronic sequence listing (JUNG-043_SEQUENCE_LISTING.txt; Size: 423 bytes; and Date of Creation: Oct. 13, 2023) is herein incorporated by reference in its entirety.

### BACKGROUND ART

RAS is a mutated proto-oncogene in human cancer, and RAS proteins are encoded by HRAS, KRAS, and NRAS genes. HRAS, KRAS, and NRAS proteins are small GTPases that act as master regulators of a signaling system involved in various cellular processes such as cell differentiation, growth, and apoptosis. The small GTPase RAS protein acts as a “molecular switch” that fluctuates between inactive and active states, and may be activated by exchange of guanosine triphosphate (GTP), which is catalyzed by a guanine nucleotide exchange factor. On the contrary, the GTP is hydrolyzed to GDP to be inactivated when catalyzed by GTPase-activated proteins (GAPs).

Activated mutants of RAS are found in specific human cancer. Approximately 9 to 30% of human tumors have RAS-activating mutants, which commonly occur in KRAS (86%), NRAS (11%), and HRAS (3%). Among them, KRAS has been a target of a drug design for over 30 years due to the most frequently mutated oncogene in human cancers such as pancreatic cancer (90%), colorectal cancer (40%), and non-small cell lung cancer (20%). Cancers with RAS mutants are aggressive and do not respond well to standard therapies. To date, drugs that successfully target RAS mutant genes have been not designed, and these mutants make the RAS protein insensitive to GTP-induced hydrolysis by GTP to fix the protein to an active state. That is, it is difficult to directly target the oncogene due to a very low affinity of the drug to the KRAS mutants. So far, potential inhibitory molecules have been reported to target RAS functional interactions indirectly without binding to RAS.

The most common KRAS mutant types are G12C, G12D, and G12V, accounting for 83% of all KRAS mutants. Among them, ovarian carcinoma patients with the KRAS G12V mutant had shorter overall survival periods than patients without the mutant. For these reasons, selectively targeting the KRAS G12V mutant is one of the first targets for ovarian cancer treatment. The number of mice causing lymph node metastasis was higher in KRAS G12V (73%) and KRAS G13D (29%) than in KRAS WT (11%) mice. Therefore, inhibitors should induce accumulation of inactive Ras by shifting the relative nucleotide affinity of Ras to prefer GDP to GTP. So far, small molecules bound to RAS have shown no nucleotide preference.

### DISCLOSURE

### Technical Problem

Accordingly, the present inventors have developed an anticancer peptide for directly targeting mutant KRAS while studying a method for directly targeting RAS mutants and then completed the present invention.

An object of the present invention is to provide an anticancer peptide represented by an amino acid sequence set forth in SEQ ID NO: 1 and a composition including the anticancer peptide.

Another object of the present invention is to provide a method for prevention or treatment of cancer, including treating an anticancer peptide represented by an amino acid sequence set forth in SEQ ID NO: 1 to a subject in need thereof.

### Technical Solution

One aspect of the present invention provides an anticancer peptide represented by an amino acid sequence set forth in SEQ ID NO: 1.

Another aspect of the present invention provides a pharmaceutical composition for prevention or treatment of cancer including an anticancer peptide represented by an amino acid sequence set forth in SEQ ID NO: 1.

Yet another aspect of the present invention provides a food composition for prevention or amelioration of cancer including an anticancer peptide represented by an amino acid sequence set forth in SEQ ID NO: 1.

Still another aspect of the present invention provides an anticancer adjuvant composition including an anticancer peptide represented by an amino acid sequence set forth in SEQ ID NO: 1.

Still yet another aspect of the present invention provides a method for prevention or treatment of cancer, including treating an anticancer peptide represented by an amino acid sequence set forth in SEQ ID NO: 1 to a subject in need thereof.

### Advantageous Effects

According to the present invention, the mutant KRAS-targeting peptide has effects of not only forming a conjugate with mutant KRAS, but also inhibiting cancer by blocking the activity of RAS, and thus can be used in various ways in the fields of cancer prevention and treatment.

### DESCRIPTION OF DRAWINGS

FIG. 1 is a diagram illustrating a result of predicting a 3D structural model of an anticancer peptide GJ101 according to the present invention.

FIG. 2 is a diagram illustrating a result of predicting a 3D structural model of binding of the anticancer peptide GJ101 according to the present invention and a KRAS G12V protein.

FIG. 3 is a diagram illustrating a result of evaluating the affinity of the anticancer peptide GJ101 to KRAS G12V through isothermal titration calorimetry analysis.

FIG. 4 is a diagram illustrating a result of confirming GTP inhibitory activity of the anticancer peptide GJ101 through guanine nucleotide binding assay.

FIG. 5 is a diagram illustrating a result of confirming an effect of the anticancer peptide GJ101 on the cell viability of cancer cells through MTT assay.

FIG. 6 is a diagram illustrating a result of confirming body weight changes in a cancer animal model administered with the anticancer peptide GJ101 according to the present invention.

FIG. 7 is a diagram illustrating a result of measuring tumor volumes in a cancer animal model administered with the anticancer peptide GJ101 according to the present invention.

FIG. 8 is a diagram illustrating a result of measuring tumor weights obtained from a cancer animal model administered with the anticancer peptide GJ101 according to the present invention.

### BEST MODE OF THE INVENTION

Hereinafter, the present invention will be described in detail.

According to an aspect of the present invention, the present invention provides an anticancer peptide represented by an amino acid sequence set forth in SEQ ID NO: 1.

In the present invention, the peptide refers to a linear molecule formed by binding amino acid residues to each other by peptide bonds. The peptide may be prepared according to a chemical synthesis method known in the art, and preferably prepared according to a solid-phase synthesis technique, but is not limited thereto.

In an embodiment of the present invention, the anticancer peptide is derived from H-REV107, which is a growth inhibitory RAS target gene capable of inhibiting anchorage-independent growth in vivo, and is preferably represented by an amino acid sequence set forth in SEQ ID NO: 1. Since the anticancer peptide consists of 5 amino acids in short length, there is an advantage of being easy to mass-produce and commercially available, and has a remarkably high binding affinity to mutant KRAS to have specificity in sequence selection.

The anticancer peptide according to the present invention includes functional equivalents and salts thereof.

In the present invention, the functional equivalents refer to peptides which have sequence homology (that is, identity) of at least 80% or more, preferably 90%, more preferably 95% or more with the peptide represented by SEQ ID NO: 1, for example, sequence homology of 80%, 81%, 82%, 83%, 84%, 85%, 86%, 87%, 88%, 89%, 90%, 91%, 92%, 93%, 94%, 95%, 96%, 97%, 98%, 99%, and 100% as a result of addition, substitution, or deletion of amino acids, and exhibit substantially homogeneous physiological activity as the peptide represented by SEQ ID NO: 1. In the present specification, the sequence homology and homogeneity are defined as a percentage of amino acid residues of a candidate sequence to the amino acid sequence set forth in SEQ ID NO: 1 after aligning the amino acid sequence set forth in SEQ ID NO: 1 and the candidate sequence and introducing gaps. If necessary, conservative substitution is not considered as part of sequence homogeneity in order to obtain the maximum percentage sequence homogeneity. An N-terminal, a C-terminal or internal extension, or deletion or insertion of the amino acid sequence set forth in SEQ ID NO: 1 is not construed as a sequence affecting sequence homology or homogeneity.

In addition, the sequence homogeneity may be determined by a general standard method used to compare similar portions of amino acid sequences of two polypeptides. A computer program such as BLAST or FASTA aligns the two polypeptides so as to optimally match respective amino acids (according to a full-length sequence of one or two sequences, or a predicted portion of one or two sequences). The program provides a default opening penalty and a default gap penalty and provides a scoring matrix such as PAM250 (standard scoring matrix; Dayhoff et al., in Atlas of Protein Sequence and Structure, vol 5, supp 3, 1978) which may be associated and used together with the computer program. For example, the percentage homogeneity may be calculated as follows. The total number of identical matches is multiplied by 100 and then divided into a sum of the length of a longer sequence in a corresponding matched span and the number of gaps introduced into the longer sequence to align the two sequences.

In the present invention, the substantially homogeneous physiological activity refers to anticancer activity. The scope of the functional equivalents of the present invention includes derivatives in which some chemical structures of the peptide are modified while maintaining the backbone and anticancer activity of the peptide represented by SEQ ID NO: 1. For example, the scope of the functional equivalents includes structural modifications for changing the stability, storage, volatility, solubility, or the like of the peptide.

In an embodiment of the present invention, the anticancer peptide may inhibit the formation of a conjugate of mutant KRAS and H-REV107.

In an embodiment of the present invention, the anticancer peptide forms a conjugate with mutant KRAS G12V, but is not limited thereto.

In the present invention, the cancer means a generic term for diseases caused by cells with aggressive characteristics in which cells divide and grow regardless of normal growth limits, invasive characteristics of infiltrating surrounding tissues, and metastatic characteristics of spreading to other parts of the body.

In an embodiment of the present invention, the cancer is preferably at least one selected from the group consisting of gastric cancer, breast cancer, lung cancer, liver cancer, blood cancer, bone cancer, pancreatic cancer, skin cancer, head or neck cancer, cutaneous or intraocular melanoma, uterine sarcoma, ovarian cancer, rectal cancer, anal cancer, colon cancer, fallopian tube carcinoma, endometrial carcinoma, cervical cancer, small intestine cancer, endocrine cancer, thyroid cancer, parathyroid cancer, adrenal cancer, soft tissue tumor, urethral cancer, prostate cancer, bronchogenic cancer, and bone marrow tumor, but is not limited thereto.

The anticancer peptide according to the present invention has effects of not only forming a conjugate with mutant KRAS, but also inhibiting the formation of a conjugate with H-REV107 by blocking the activity of RAS, that is, suppressing cancer, and thus can be used in various ways in the field of prevention, amelioration, or treatment of cancer.

According to another aspect of the present invention, the present invention provides a pharmaceutical composition for prevention or treatment of cancer including an anticancer peptide represented by an amino acid sequence set forth in SEQ ID NO: 1.

When the composition of the present invention is used as a pharmaceutical composition, the pharmaceutical composition of the present invention may be formulated and used in various forms according to conventional methods. For example, the pharmaceutical composition may be prepared for oral formulations, such as powders, granules, tablets, capsules, suspensions, emulsions, syrups, and the like, and formulated and used in the form of external preparations, suppositories, and sterile injection solutions.

The composition of the present invention may contain at least one known active ingredient having a preventive or therapeutic effect on cancer together with the anticancer peptide.

The composition of the present invention may further include a pharmaceutically acceptable additive. At this time, the pharmaceutically acceptable additive may be used with starch, gelatinized starch, microcrystalline cellulose, lactose, povidone, colloidal silicon dioxide, calcium hydrogen phosphate, lactose, mannitol, syrup, arabic gum, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, white sugar, and the like. The pharmaceutically acceptable additive according to the present invention is preferably included in an amount of 0.1 to 90 parts by weight based on the composition, but is not limited thereto.

The composition of the present invention may be administered in various oral or parenteral formulations during actual clinical administration, but may be prepared using commonly used diluents or excipients, such as fillers, extenders, binders, wetting agents, disintegrants, surfactants, etc., for formulations. Suitable formulations known in the art are preferably used with formulations disclosed in the literature (Remington's Pharmaceutical Science, last, Mack Publishing Company, Easton PA).

Solid formulations for the oral administration include tablets, pills, powders, granules, capsules, and the like, and the solid formulations may be prepared by mixing at least one excipient, for example, starch, calcium carbonate, sucrose or lactose, gelatin, and the like. Further, lubricants such as magnesium stearate and talc may be used in addition to simple excipients. In addition, liquid formulations for the oral administration may correspond to suspensions, oral liquids, emulsions, syrups, and the like, and may include various excipients, for example, wetting agents, sweeteners, aromatic agents, preservatives, and the like, in addition to water and liquid paraffin which are commonly used as simple diluents.

The formulations for the parenteral administration include a sterile aqueous solution, a non-aqueous solution, a suspension, an emulsion, a lyophilizing agent, and a suppository. As the non-aqueous solution and the suspension, propylene glycol, polyethylene glycol, vegetable oil such as olive oil, injectable ester such as ethyl oleate, and the like may be used. As a base of the suppository, witepsol, macrogol, tween 61, cacao butter, laurinum, glycerogelatin, and the like may be used.

The dose of the pharmaceutical composition of the present invention may vary depending on a formulation method, an administration method, an administration time, and/or an administration route of the pharmaceutical composition. In addition, the dose may vary depending on various factors including the type and degree of a response to be achieved by administration of the pharmaceutical composition, the type, age, body weight, general health condition, symptoms or severity of a disease, sex, diet, and excretion of a subject to be administered, drugs used simultaneously or separately for the corresponding subject, other composition ingredients, and the like, and similar factors well-known in the field of medicine. An effective dose for a desired treatment may be easily determined and prescribed by those skilled in the art.

The administration route and the administration method of the pharmaceutical composition of the present invention may be independent of each other, and the method is not particularly limited, and may be any administration route and administration method so long as the pharmaceutical composition may reach a target site.

The pharmaceutical composition of the present invention may be used alone or in combination with surgery, radiation therapy, hormone therapy, chemotherapy, and methods of using biological response modifiers for prevention or treatment of cancer.

According to yet another aspect of the present invention, the present invention provides a food composition for prevention or amelioration of cancer including an anticancer peptide represented by an amino acid sequence set forth in SEQ ID NO: 1.

When the composition of the present invention is used as a food composition, the food composition of the present invention means foods having an effect of preventing or ameliorating cancer and diseases caused by cancer, and should be harmless to the human body when taken for a long period of time.

The kind of food is not particularly limited. Examples of the food which may be added with the materials include meat, sausages, bread, chocolate, candies, snacks, confectionery, pizza, ramen, other noodles, gums, dairy products including ice cream, various soups, beverages, tea, drinks, alcohol drinks, vitamin complex, and the like, and include all health functional foods in an accepted meaning.

In an embodiment of the present invention, the food composition of the present invention may be a food additive. The food additive may be added with the anticancer peptide as it is or used with other foods or food ingredients, and may be appropriately used according to general methods. The mixed amount of the active ingredients may be suitably determined according to a purpose of use (prevention, health, or therapeutic treatment).

In the embodiment of the present invention, the food composition of the present invention may be a health drink composition. The health drink composition may include various flavoring agents, natural carbohydrates, or the like as an additional ingredient like general drinks, in addition to the anticancer peptide. As the above-mentioned natural carbohydrates, monosaccharides such as glucose and fructose, disaccharides such as maltose and sucrose, natural sweeteners such as dextrin and cyclodextrin, synthetic sweeteners such as saccharin and aspartame, and the like may be used. A ratio of the natural carbohydrates may be generally about 0.01 to 10 g, preferably about 0.01 to 0.1 g per 100 ml of the composition of the present invention.

In addition to the ingredients, the composition of the present invention may include various nutrients, vitamins, electrolytes, flavoring agents, coloring agents, pectic acid and salts thereof, alginic acid and salts thereof, organic acid, a protective colloidal thickener, a pH adjusting agent, a stabilizer, a preservative, glycerin, alcohol, a carbonating agent used in a carbonated drink, or the like. In addition, the composition of the present invention may include pulps for preparing natural fruit juices, fruit juice beverages, or vegetable beverages. These ingredients may be used independently or in combination. Although the ratio of these additives is not greatly important, generally, the ratio thereof is selected in a range of 0.01 to 0.1 part by weight per 100 parts by weight of the composition of the present invention.

In addition, the present invention provides an anticancer adjuvant composition including an anticancer peptide represented by an amino acid sequence set forth in SEQ ID NO: 1.

In the present invention, the anticancer adjuvant refers to an agent that may be used adjunctively to enhance an effect of a cancer therapeutic agent commonly used in the art, and the adjuvant according to the present invention is used to enhance the effect of a cancer therapeutic agent or anticancer treatment.

The anticancer adjuvant composition of the present invention may be in the form of a pharmaceutical composition or food composition, and more specifically, an anticancer pharmaceutical adjuvant or anticancer food adjuvant.

According to still yet another aspect of the present invention, the present invention provides a method for prevention or treatment of cancer, including treating an anticancer peptide represented by an amino acid sequence set forth in SEQ ID NO: 1 to a subject in need thereof.

In an embodiment of the present invention, the subject may be a subject expected to develop cancer; a subject suffering from cancer; or a subject completely cured of cancer, but is not limited thereto.

### MODES OF THE INVENTION

Hereinafter, the present invention will be described in more detail through Examples. These Examples are just illustrative of the present invention, and it will be apparent to those skilled in the art that it is not interpreted that the scope of the present invention is limited to these Examples.

### Example 1. Discovery of Mutant KRAS-Targeting Anticancer Peptide

Since conventionally developed cancer therapeutic agents have very low affinity for mutant Kirsten-RAS (KRAS), it was difficult to directly target the oncogene. In addition, since a mechanism related to the affinity of the mutant KRAS has not been identified, it was difficult to determine its structure. Therefore, studies on oncogenic mutant KRAS and an HRAS-like suppressor 3 (H-REV107) conjugate were conducted. As a result, the interaction between the mutant KRAS and the H-REV107 conjugate was molecularly modeled, and based on the result, a mutant KRAS-targeting anticancer peptide was designed. The designed anticancer peptide was represented by an amino acid sequence set forth in SEQ ID NO: 1, and was named as ‘GJ101’.

The anticancer peptide GJ101 was produced using Fomc solid phase peptide synthesis (SPPS) and purified by reverse phase high performance liquid chromatography (RP-HPLC) with a purity of 95% or more. The purified anticancer peptide GJ101 was identified using liquid chromatography/mass spectrometry (LC-MS).

### Example 2. Prediction of 3D Structural Model of Anticancer Peptide GJ101

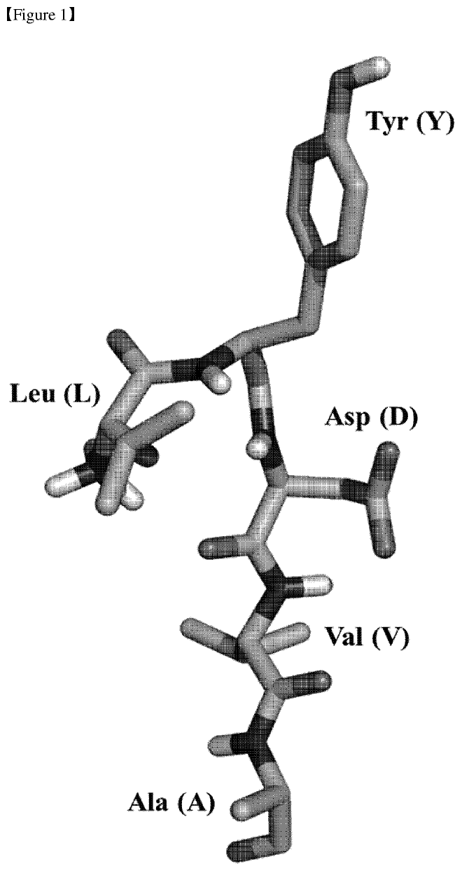

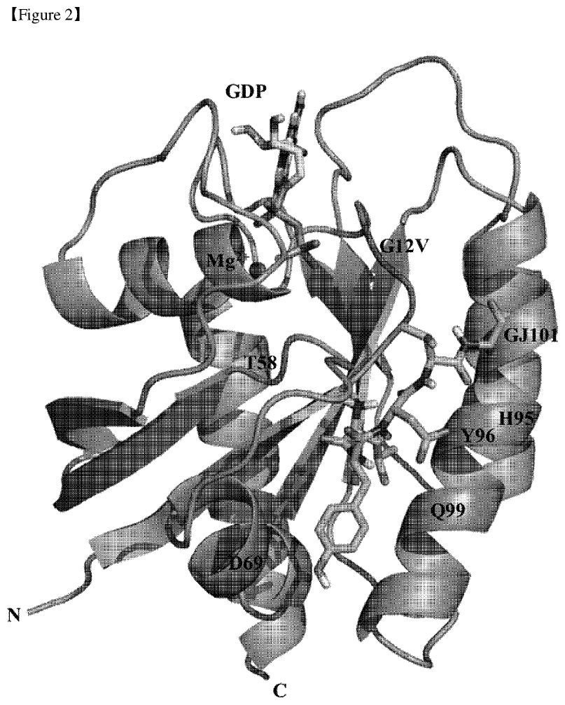

To determine the binding between a KRAS G12V protein and GJ101, a conjugate model was predicted in a 3D structure. To this end, a 3D protein structural model which has been recently identified by the present inventors was used, and a virtual screening tool was used as a predictive model. The result of predicting the 3D structural model of the anticancer peptide GJ101 was illustrated in FIG. 1, and the result of predicting the binding structure of the anticancer peptide GJ101 and the KRAS G12V protein was illustrated in FIG. 2.

As illustrated in FIG. 1, a 3D structural model of the anticancer peptide GJ101 was confirmed.

As illustrated in FIG. 2, it was confirmed that the anticancer peptide GJ101 formed hydrogen bonds with five amino acid residues T58, D69, H95, Y96, and Q99 of the KRAS G12V protein, and its binding affinity was—5.1 kcal/mol.

### Example 3. Evaluation of Affinity of Anticancer Peptide GJ101 to KRAS G12V

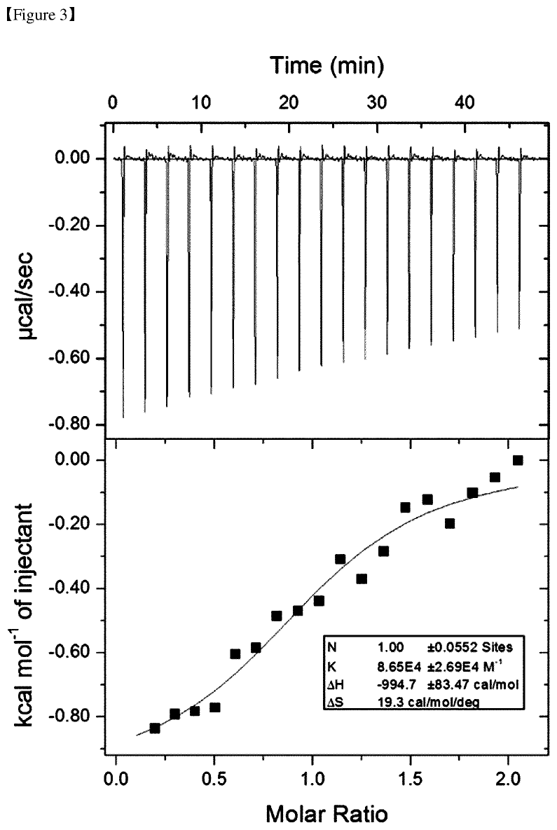

In order to examine the affinity of the anticancer peptide GJ101 to the KRAS G12V protein, isothermal titration heat was measured using isothermal titration calorimetry (ITC). Specifically, the KRAS G12V protein was prepared by dialysis with a buffer [20 mM Tris-HCl (pH 7.4), 100 mM NaCl, and 2 mM MgCl₂] so that the final concentration of the KRAS G12V protein was 0.1 mM. Next, the anticancer peptide GJ101 was prepared by diluting the anticancer peptide GJ101 to a concentration of 1.0 mM using the same buffer as described above. Isothermal titration heat measurement was performed by injecting the prepared KRAS G12V protein and anticancer peptide GJ101 into isothermal titration calorimetry. In the isothermal titration heat measurement, the anticancer peptide GJ101 was injected 20 times at 25° C. while stirring at a rate of 1,000 rpm. ΔS was calculated from a standard thermodynamic equation using the calculated K and ΔH values. The results of isothermal titration calorimetry were illustrated in FIG. 3.

As illustrated in FIG. 3, the K and ΔH values calculated through isothermal titration calorimetry were 8.65E4±2.69E4 M⁻¹ and −994.4±83.47 cal/mol, respectively, and the ΔS value calculated using these values was 19.3 cal/mol/deg. In addition, it was confirmed that the binding affinity (K_{D}) of the anticancer peptide GJ101 to the KRAS G12V protein was 12 μM. The results indicated that the anticancer peptide GJ101 formed a strong interaction with the KRAS G12V protein and may form a stable conjugate capable of blocking an RAS activation function.

### Example 4. Evaluation of GTP Inhibitory Activity of Anticancer Peptide GJ101

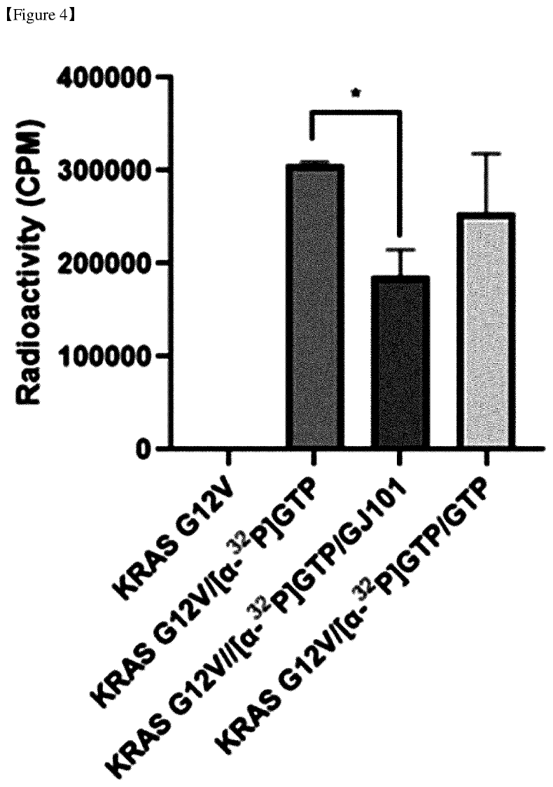

In order to confirm whether the anticancer peptide GJ101 inhibited the activity of GTP, guanine nucleotide binding assay was performed. Specifically, a recombinant gene KRAS G12V cloned into a vector pET28a was inserted into E. coli BL21 (DE3) and expressed. The overexpressed protein was purified using affinity chromatography and gel filtration chromatography. The purified KRAS G12V protein was bound to Ni-NTA beads at 4° C. using a binding buffer [50 mM Hepes (pH 7.5), 100 mM NaCl, 2 mM MgCl₂, 1 mM EDTA, and 1 mM DTT]. The Ni-NTA beads loaded with the bound KRAS G12V protein were washed with a binding buffer, treated with radioactive isotopes [(α-³²P] GTP (2,500 cpm/pmol) and GJ101 at 37° C., and then reacted. A control group was an untreated group (KRAS G12V); a [α-³²P] GTP-treated group (KRAS G12V/[α-³²P] GTP); and a [α-³²P] GTP and GTP-treated group (KRAS G12V/[α-³²P] GTP/GTP). Then, the Ni-NTA beads were then washed with a wash buffer [20 mM Tris-HCl (pH 7.4), 100 mM NaCl, and 2 mM MgCl₂]. The bead-bound KRAS G12V protein was eluted using 200 mM Imidazole, and radioactive nucleotides were quantified by liquid scintillation counting. The result of the guanine nucleotide binding assay was illustrated in FIG. 4.

As illustrated in FIG. 4, it was confirmed that in the absence of the anticancer peptide GJ101, the KRAS G12V protein and [α-³²P] GTP had high binding strength, but in the presence of the anticancer peptide GJ101, the binding strength of KRAS G12V and [α-³²P] GTP was decreased by about 40%. The result indicated that the anticancer peptide GJ101 interacted with KRAS G12V to inhibit the GTP activity.

### Example 5. Examination of Effect of Anticancer Peptide GJ101 on Cell Viability of Cancer Cells

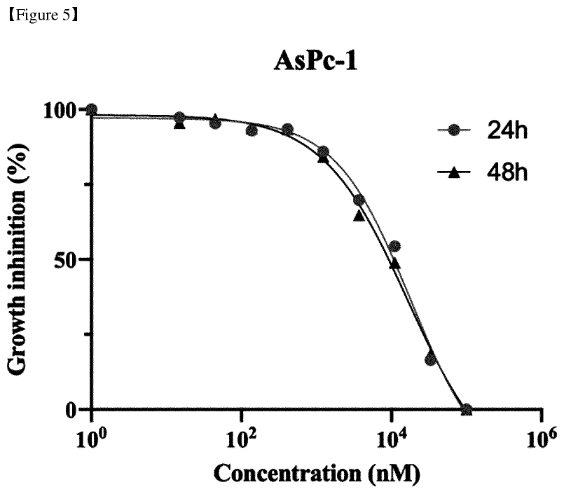

Through 3-(4,5-dimethylthiazol-2-yl)-2,5-diphenyltetrazolium bromide (MTT) analysis, an effect of the anticancer peptide GJ101 on the cell viability of cancer cells was examined. For the cell viability analysis, AsPC-1 cells as a pancreatic cancer cell line were used. The pancreatic cancer cell line was prepared by dispensing the pancreatic cancer cell line at 1×10³ cells/well (100 μL) and adhering thereto. To treat the anticancer peptide GJ101, the medium of the cells was replaced with a new medium (RPMI-1640). The pancreatic cancer cell lines with different adhering times were treated with the anticancer peptide GJ101 while increasing a concentration gradient, respectively. The pancreatic cancer cells treated with the anticancer peptide GJ101 were incubated for 24 or 48 hours, respectively. Each incubated pancreatic cancer cell was added with an MTT solution (5 mg/mL) and incubated for 90 minutes. After the end of the incubation, an optical density (OD) of pancreatic cancer cells was measured at 540 nm using a microplate reader (Molecular Devices, USA), and a maximum concentration (GI₅₀) at which the growth of pancreatic cancer cells was reduced by half was confirmed. The result of examining the effect of the anticancer peptide GJ101 on the cell viability of tumor cells was illustrated in FIG. 5.

As illustrated in FIG. 5, it was confirmed that the anticancer peptide GJ101 decreased the cell viability in a time- and dose-dependent manner. In addition, as a result of measuring the GI₅₀ value, it was confirmed that the anticancer peptide GJ101 effectively inhibited the growth of pancreatic cancer cells even at a very low concentration of 16 μM.

### Example 6. Evaluation of Activity of Anticancer Peptide GJ101 in Cancer Animal Model

6-1. Preparation of Cancer Animal Model

To prepare a cancer animal model, AsPC-1 cells as a pancreatic cancer cell line were prepared by dissociation in a Hanks' balanced salt solution (HBSS) and a materigel. The prepared AsPC-1 cells were transplanted into the right flank of a nude mouse at an amount of 2×10⁶ cells/10 μL/animal to prepare a cancer animal model.

For an experiments to be described later, the anticancer peptide GJ101 was administered at a concentration of 25 mg/kg or 50 mg/kg from day 16 after the preparation of the cancer animal model. In addition, a control group (Vehicle) was treated with 10% DMSO, 40% PEG400, and 50% DW.

6-2. Breeding Conditions

Each experimental animal was given a unique animal number according to group separation and individual, and the animal number was marked on the mouse's right ear. After the mice were obtained, 3 to 5 mice per cage were housed in individually ventilated cage system (IVCS)-based cages (391W×199D×160H mm) in No. 609 in the re-entry area on the 2nd floor of the Experimental Animal Center. The temperature of a breeding room was maintained at 22±1° C., the relative humidity was 50±10%, and the number of ventilation times was 10 to 15 times/hr. A light-dark cycle of the breeding room was maintained at 12 hours/day (07:00 to 19:00) and observed. The illumination of the breeding room was maintained at 150 to 300 Lux. A mouse feed was received from Woojung Bio Co., Ltd., to confirm monitoring information, and a solid feed was put in a feeder and freely consumed. In the case of drinking water, the water supply of Daegu was purified through an RO water device and then freely consumed.

6-3. Measurement of Body Weight in Cancer Animal Model

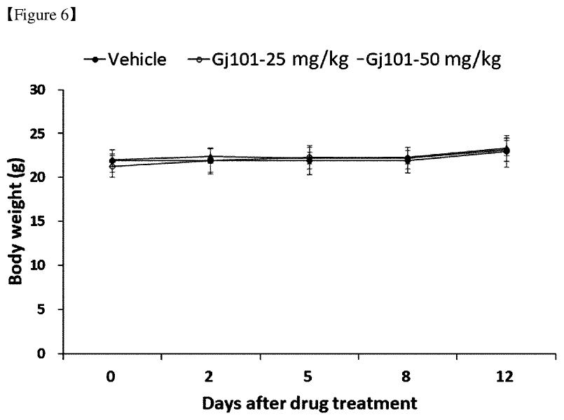

A drug was administered to a cancer animal model, and then the body weight of the individual was measured once a day, and the result were illustrated in FIG. 6.

As illustrated in FIG. 6, it was confirmed that there was no significant body weight change in the anticancer peptide GJ101-treated group and the control group. In addition, in the cancer animal model, no change in body weight was observed according to pancreatic cancer cell line transplantation and drug administration, and a moribund state or behavioral abnormalities were not observed.

6-4. Measurement of Tumor Volume

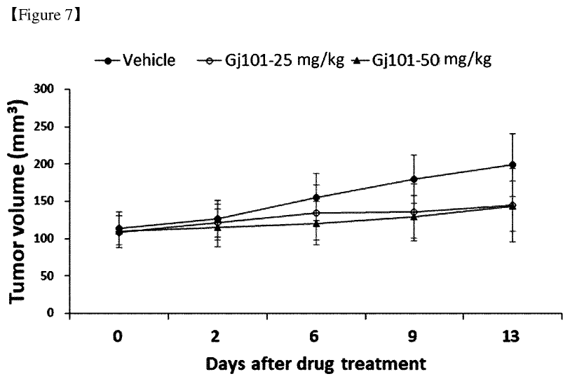

The drug was administered to the cancer animal model, and then the tumor volume was measured, and the measured value was analyzed by a student's t-test. The result of measuring the tumor volume was illustrated in FIG. 7.

As illustrated in FIG. 7, it was confirmed that the tumor volume of the control group was increased significantly from 2 days after administration, whereas the tumor volume of the anticancer peptide GJ101-treated group was not increased.

6-5. Measurement of Tumor Weight

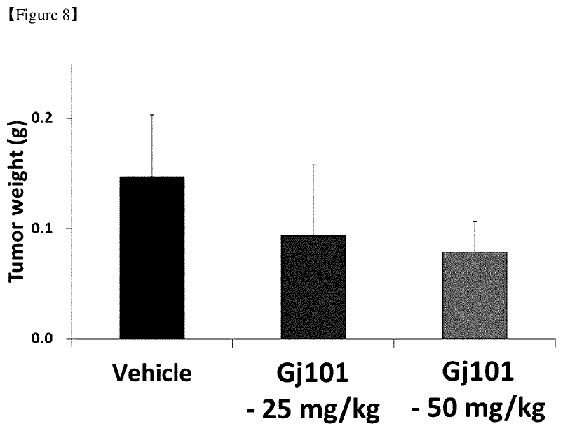

After the experiment of Example 6-4 was completed, the cancer animal model was sacrificed. Thereafter, tumors were excised from the sacrificed cancer animal model, and the weights thereof were measured. From the measured values, an average weight of the tumor tissue of each individual was calculated, which was comprehensively analyzed through student's t-test and dunnett's test. The result of measuring the tumor weight was illustrated in FIG. 8.

As illustrated in FIG. 8, it was confirmed that the tumor weights of the anticancer peptide GJ101 25 and 50 mg/kg treated groups were decreased by 36% and 46%, respectively, compared to the control group.

The result means that there is a significant anticancer effect in a cancer animal model, that is, an AsPC-1 xenograft animal model.

Overall, the present inventors developed the anticancer peptide GJ101, and confirmed that the anticancer peptide GJ101 not only formed a conjugate with mutant KRAS but also blocked the activity of RAS. This means that the anticancer peptide GJ101 has cancer inhibitory activity, and can be used in various ways in the fields of cancer prevention and treatment.

Hereinafter, the present invention will be described in more detail with reference to Preparation Examples. Preparation Examples are only illustrative of the present invention, and the scope of the present invention is not construed to be limited to Preparation Examples.

### Preparation Example 1. Preparation of Pharmaceutical Composition for Prevention or Treatment of Cancer

1-1. Preparation of Powders

Anticancer peptide represented by amino acid sequence set forth in SEQ ID NO. 1: 1 mg

Lactose: 100 mg

Talc: 10 mg

The ingredients were mixed and filled in an airtight bag to prepare powders.

1-2. Preparation of Tablets

Anticancer peptide represented by amino acid sequence set forth in SEQ ID NO. 1: 1 mg

Corn starch: 100 mg

Lactose: 100 mg

Magnesium stearate: 2 mg

The ingredients were mixed and then tableted according to a general tablet preparation method to prepare tablets.

1-3. Preparation of Capsules

Anticancer peptide represented by amino acid sequence set forth in SEQ ID NO. 1: 1 mg

Crystalline cellulose: 3 mg

Lactose: 14.8 mg

Magnesium stearate: 0.2 mg

The ingredients were mixed and filled into gelatin capsules according to a general capsule preparation method to prepare capsules.

1-4. Preparation of Injections

Anticancer peptide represented by amino acid sequence set forth in SEQ ID NO. 1: 1 mg

Mannitol: 180 mg

Sterile distilled water for injection: 2974 mg

Na₂HPO₄2H₂O: 26 mg

The injections were prepared with the ingredient content per 1 ampoule (2 ml) according to a general method for preparing injections.

1-5. Preparation of Liquids

Anticancer peptide represented by amino acid sequence set forth in SEQ ID NO. 1: 1 mg

Isomerized sugar: 10 g

Mannitol: 5 g

Purified water: Suitable amount

According to a general method of preparing liquids, each ingredient was added and dissolved in purified water, added with lemon flavor in an appropriate amount, and then the ingredients were mixed, added with purified water to be adjusted to a total of 100 ml, and then filled in a brown bottle and sterilized to prepare liquids.

As described above, specific parts of the present invention have been described in detail, and it will be apparent to those skilled in the art that these specific techniques are merely preferred embodiments, and the scope of the present invention is not limited thereto. Therefore, the substantial scope of the present invention will be defined by the appended claims and their equivalents.

1. An anticancer peptide represented by amino acid sequence set forth in SEQ ID NO: 1.

2. The anticancer peptide of claim 1, wherein the anticancer peptide is derived from H-REV107.

3. The anticancer peptide of claim 1, wherein the anticancer peptide inhibits formation of a conjugate of mutant Kirsten-RAS (KRAS) and HRAS-like suppressor 3 (H-REV107).

4. The anticancer peptide of claim 1, wherein the anticancer peptide forms a conjugate with a mutant KRAS G12V.

5. The anticancer peptide of claim 1, wherein the cancer is at least one selected from the group consisting of gastric cancer, breast cancer, lung cancer, liver cancer, blood cancer, bone cancer, pancreatic cancer, skin cancer, head or neck cancer, cutaneous or intraocular melanoma, uterine sarcoma, ovarian cancer, rectal cancer, anal cancer, colon cancer, fallopian tube carcinoma, endometrial carcinoma, cervical cancer, small intestine cancer, endocrine cancer, thyroid cancer, parathyroid cancer, adrenal cancer, soft tissue tumor, urethral cancer, prostate cancer, bronchogenic cancer, and bone marrow tumor.

6-9. (canceled)

10. A method for treatment or amelioration of cancer, the method comprising step of administering a composition comprising a peptide represented by the amino acid sequence of SEQ ID NO: 1.

11. The method of claim 10, wherein the composition is a pharmaceutical composition, food composition or anticancer adjuvant composition.
